(19) United States
(12) Patent Application Publication
(10) Pub. No.: US 2026/0283786 A1
(43) Pub. Date: Sep. 24, 2026

(54) ACCOMMODATIVE INTRAOCULAR LENS SYSTEM AND SET FOR ASSEMBLING THE ACCOMMODATIVE INTRAOCULAR LENS SYSTEM.

(71) Applicant: Rayner Intraocular Lenses Limited, West Sussex (GB)

(72) Inventors: Otavio Gomes De Oliveira, Manhuaçu/Mg (BR); Matthew Clayton, Rustington (GB); Alessandro Capoia, Worthing (GB)

(73) Assignee: Rayner Intraocular Lenses Limited, West Sussex (GB)

(21) Appl. No.: 19/476,219

(22) PCT Filed: Apr. 21, 2023
(86) PCT No.: PCT/EP2023/060537

§ 371 Date: Oct. 17, 2025

(51) Int. Cl. A61F 2/16

(52) U.S. Cl. CPC A61F 2/1648; A61F 2002/1682

(57) ABSTRACT

An accommodative intraocular lens system (1) comprises a base (10) and a bonnet (4) that is attachable or attached to the base (10), wherein the base (10) includes a base lens (11) and the bonnet includes a bonnet lens (5) and a bonnet ring (7) holding the bonnet lens (5). The bonnet lens (5) is attached to the bonnet ring (7) such that when the base (10) is attached to the bonnet (4), a compression force applied to the bonnet ring (7) and to the base (10) can move the bonnet lens (5) and the base lens (11) towards each other. The base lens (11) and the bonnet lens (5) can assume at least one relative position in which the anterior face of the base lens (11) and the posterior face of the bonnet lens (5) press against each other forming a contact area. At least one of the bonnet (4) and the base (10) comprises a stop (17) that protrudes from the one towards the other of the bonnet (4) and the base (10) and that can abut against the other of the bonnet (4) and the base (10) in order to limit the extent by which the anterior face of the base lens (11) and the posterior face of the bonnet lens (5) can be moved towards each other to press against each other. At least one of the base (10) and the bonnet (4) is provided with elastic webs (13) that render the bonnet lens (5) movable relatively to the base lens (11). At the interface of at least one of the webs (13) with the base (10) or the bonnet (4) a notch (16) is provided. The base lens is provided with a foot (14). A set comprises multiple bases (10) and a bonnet (4) that is attachable to each of the bases (10).

### FIELD OF THE INVENTION

The invention relates to an accommodative intraocular lens system comprising a base component and a bonnet that is attachable or attached to the base, the bonnet and the base each comprising a lens, the lenses being movable relatively to each other. Moreover, the invention relates to a set for assembling the accommodative intraocular lens system. The invention also relates to a method of operation of such accommodative intraocular lens system and a method of assembling the accommodative intraocular lens system.

### Background of the Invention

The natural lens of the eye is soft and resilient, and therefore can change its optical power and thus its focal length as the ciliary muscles impose varying forces on the lens via the zonules and the capsular bag in which the lens sits. By changing the focal length, generally referred to as “accommodation”, the lens can have a short focal length for near vision and a long focal length for distance vision.

The natural lens may become cloudy, generally referred to as a “cataract”. Cataracts are most commonly due to aging but may also occur due to trauma or be present from birth. They can be treated by surgically replacing the natural lens with an artificial lens called a pseudophakic intraocular lens. To implant the intraocular lens into the human eye, a circular hole, generally referred to as a “capsulorhexis”, is cut into the anterior side of the capsular bag. After removal of the natural lens, the intraocular lens is inserted into the emptied capsular bag through the capsulorhexis.

However, intraocular lenses are stiffer than natural lenses, and for this reason alone they cannot accommodate significantly. In addition, the inevitable damage of the capsular bag due to the capsulorhexis would interfere with accommodation, even if the intraocular lens were similarly flexible and had the same geometry as the natural lens.

In recent years, accommodating intraocular lenses have been developed that comprise two lens components and that are able to accommodate in a certain range. Typically, to mimic natural accommodation these lenses comprise two or more individual lenses or other optical components the distance between which and/or the shape of which can change thereby shifting the position of focal point along the optical axis.

US 2009/0125106 A1 discloses a two-optic accommodative lens system in which at least one of the optics is deformable. Each optic is attached to a peripheral ring by a plurality of haptics. The two rings are joined to obtain the accommodative lens system. When compressed by the capsular bag, the two optics press against each other, deforming at least one of them. As a result, the interface where the two optics meet changes shape, thereby altering the refractive power of the lens system.

From WO 2017/203517 A1 a hybrid accommodating intraocular lens system is known that comprises two separate component parts in the form of a base member for initial implantation in a vacated capsular bag and a lens unit for subsequent implantation in the vacated capsular bag for anchoring to the base member. The lens unit includes a lens with at least two haptics extending radially outwardly therefrom for fixing of the lens unit to the base member. The base member includes a flat circular base member centrepiece having zero refractive power.

EP 1 499 264 B1 describes a two-part accommodative intraocular lens comprising a first optic with negative power, a second optic with positive power and at least one haptic connected to the second optic or connected by a hinge region. The connection between the two optics can be established via a circumferential socket and locking rib.

EP 2 512 374 B1 deals with an intraocular lens system for implantation into a capsular bag of patients that suffer from age-related macular degeneration. The system comprises a first anterior lens with a positive power optic with a first optical axis and a second posterior lens with a negative power optic with a second optical axis. Both lenses include haptics that are configured to produce an offset between the first optical axis and the second optical axis.

From EP 3 049 023 B1 an accommodative intraocular lens system comprising a first optical member with variable and dynamic refractive power on the posterior side and a second ptical member with a fixed refractive power on the anterior side is known. The first member is firmly attached to the second member.

U.S. Pat. No. 4,892,543 B discloses an accommodative intraocular lens system for mounting within the ciliary body, the system comprising essentially three parts. On its anterior side a first component with a flat anterior face is provided. On the posterior side of the first component a thin pliable optical element is attached which has a normally flat rear flexible membrane and a generally forward-extending cylindrical wall which snaps into interlocking engagement with a flange on the posterior side of the first component. Behind the membrane a second component is provided that comprises a main solid central lens with a rear spherical surface seated within the vitreous body. A contraction or relaxation of the ciliary body leads to a relative movement of the second element and the membrane and thus a change in refractive power.

US 2020/0345481 A1 concerns an intraocular lens with a shape-changing optic comprising an elastic anterior face, a posterior face and an elastic side wall extending between the two faces. A chamber is located between the anterior face and the posterior face and contains a soft solid or a fluid. A similar lens is known form US 2022/0151768 A1.

### OBJECT OF THE INVENTION

It is an object of the present invention to provide an improved system comprising a base component and a front component that is attachable or attached to the base component, each components comprising a lens, the lenses being movable relatively to each other. The invention moreover aims at providing an improved set for assembling the accommodative intraocular lens system. It is a further object of the invention to provide an improved method of operation of such accommodative intraocular lens system and a method of assembling the accommodative intraocular lens system.

### Solution According to the Invention

In the following, any reference to one (including the articles “a” and “the”), two or another number of objects is, provided nothing else is expressly mentioned, meant to be understood as not excluding the presence of further such objects in the invention. The reference numerals in the patent claims are not meant to be limiting but merely serve to improve readability of the claims.

In a first aspect of the invention, an accommodative intraocular lens system according to claim 1 is provided. The system comprises a base and a bonnet that is attachable or attached to the base. The base includes a base lens and the bonnet includes a bonnet lens and a bonnet ring holding the bonnet lens. When the base is attached to the bonnet, the base lens and the bonnet lens are arranged so that they are movable relatively to each other. The bonnet lens is attached to the bonnet ring such that when the base is attached to the bonnet, a compression force applied to the bonnet ring and to the base can move the bonnet lens and the base lens towards each other.

It is achievable with this aspect of the invention that the bonnet ring can move the bonnet lens relatively to the base lens as a result of compression forces applied by the capsular bag when the accommodative intraocular lens system is implanted into the patient's eye. As a result, advantageously, the lens can respond to the forces applied by the ciliary muscles on the capsular bag through the zonules.

In the context of the present invention, a “lens” is an optical device of a material which is transparent for part of the visible spectrum, preferably a large part, more preferably essentially the entire visible spectrum of light. A lens has an anterior and a posterior face, each of which can be curved concavely, be curved convexly or be flat. The curvature of the faces determines a refractive power of the lens, which power is generally specified in dioptres. If both faces are flat or have exactly opposite curvature, the lens has a zero refractive power. The present definition of a lens includes lenses with zero refractive power.

In the context of the present invention, a “compression force” denotes an amount of a force that is applied to one component of the lens system, for example the bonnet or the bonnet lens. This amount is equal to the amount of a corresponding oppositely directed force that is applied to another component of the lens system, for example the base or the base lens.

In the following disclosure, for convenience, the side of the accommodative intraocular lens system nearer to the bonnet is arbitrarily designated the front, and accordingly, the side nearer to the base is designated as the back of the accommodative intraocular lens system.

The terms “anterior” and “posterior” are used with reference to these arbitrary designations and have the meaning common in the art. Thus, for example, the anterior face of the base lens is the face that is directed towards the front of the accommodative intraocular lens system. The terminology regarding the front and the back is merely to aid in indicating the relative position of the various components of the accommodating intraocular lens system of the invention. They do not have any meaning beyond that. In particular, they are not to be understood as requiring a particular orientation in which the lens must be implanted into the patent's eye.

In a second aspect of the invention, an accommodative intraocular lens system according to claim 8 is provided. The system comprises a base and a bonnet that is attachable or attached to the base. The base includes a base lens and the bonnet includes a bonnet lens. When the base is attached to the bonnet, the base lens and the bonnet lens are arranged so that they are movable relatively to each other. The base lens is provided with a foot.

With this aspect of the invention, it is achievable that when the base is attached to the bonnet, compression forces applied to the bonnet lens and to the foot can move the base lens and the bonnet lens towards each other. Moreover, advantageously, the foot can contribute to the lens system lying more stably in the capsular bag. In particular, the foot can be chosen so that the hybrid lens properly fits inside the capsular bag.

In a third aspect of the invention, an accommodative intraocular lens system according to claim 15 is provided. The system comprises a base and a bonnet that is attachable or attached to the base. The base includes a base lens and the bonnet includes a bonnet lens. At least one of the base and the bonnet is provided with elastic webs that render the bonnet lens movable relatively to the base lens. At the interface of at least one of the webs with the base or the bonnet a notch is provided.

It is an achievable advantage of the notch that it contributes to setting the restoring force of the web during compression to an appropriate value. Also, advantageously, the notch can contribute to concentrating strain in order to protect the base lens from unwanted deformations during compression. Such protection from deformation can increase the optical properties of the base lens, in particular in a compressed state of the lens system.

In a fourth aspect of the invention, an accommodative intraocular lens system according to claim 18 is provided. The system comprises a base and a bonnet that is attachable or attached to the base. The base includes a base lens and the bonnet includes a bonnet lens. When the base is attached to the bonnet, the base lens and the bonnet lens are arranged so that they are movable relatively to each other and the base lens and the bonnet lens can assume at least one relative position in which the anterior face of the base lens and the posterior face of the bonnet lens press against each other forming a contact area. At least one of the bonnet and the base comprises a stop that protrudes from the one towards the other of the bonnet and the base and that can abut against the other of the bonnet and the base in order to limit the extent by which the anterior face of the base lens and the posterior face of the bonnet lens can be moved towards each other to press against each other.

With the stop attached to one of the bonnet and the base, it is achievable that that when the stop is touched by the edge of the other of the bonnet and the base, it can limit the force with which the bonnet lens and the base lens are pressed against each other. In other words, the stop can resist to further unwanted compression. This way, it can be achieved that in the compressed state, the bonnet and the base lens are pressed against each other with a well-defined force. The well-defined force can entail a well-defined deformation of the bonnet lens and the base lens.

In a fifth aspect of the invention, a set for assembling an accommodative intraocular lens system suitable for implantation in capsular bags of multiple sizes according to claim 22 is provided. The set comprises multiple bases and a bonnet that is attachable to each of the bases. The bonnet has an actuator for contacting a portion of the capsular bag on one of the anterior and the posterior sides of the capsular bag, and each base has an actuator for contacting a portion of the capsular bag on the other of the anterior and the posterior sides of the capsular bag. Each base is shaped differently such that for each base when the base is attached to the bonnet, the length of the resulting accommodative intraocular lens as measured from the actuator of the base to the actuator of the bonnet is different to that of any accommodative intraocular lens formed with any of the other bases.

It is an achievable advantage of the set according to the invention that a base can be chosen from the multiple bases so that the hybrid lens properly fits inside the capsular bag. This can help to ensure proper response of the lens system to the forces applied via the capsular bag by the ciliary muscles through the zonules. In particular, with this aspect of the invention, it can be avoided that the accommodative intraocular lens system is too small or too large for a given capsular bag. The lens system being too small for the capsular bag could entail that it cannot be compressed enough by the patient's eye to produce distance vision. The lens system being too large for the capsular bag could entail that it can be permanently compressed with the result that the patient would not experience near vision.

In the context of the present invention, an actuator is a part of a component of the accommodative intraocular lens system, for example of the bonnet ring or the foot, that is intended to contact the capsular bag when the accommodative intraocular lens system is implanted in the capsular bag.

Whenever herein an intraocular lens system or an embodiment of an intraocular lens system or a component of such system is disclosed, this is meant to disclose also an embodiment of the set for assembling an accommodative intraocular lens system which embodiment comprises such system or component. In particular, whenever an embodiment of a base is disclosed, this is meant to disclose also an embodiment of the set wherein at least one, preferably all of the bases of the set are such base.

When in the context of the present invention dimensions, the shape or the refractive power of the accommodative intraocular lens system or its components-such as the dimensions indicated above or the shape or refractive power of the faces of the lens—are recited, these are measured in the relaxed state of the system or the component at issue. The relaxed state is the state in which no external force—such as a force originating from the capsular bag—acts upon the accommodative intraocular lens system or the component at issue. As a result, the dimensions, shapes and refractive powers for example of the base lens and the bonnet lens are measured in a state in which they do not press against each other or are compressed by other forces.

In a sixth aspect of the invention, a method of operation of an accommodative intraocular lens system according to claim 25 is disclosed. The system comprises a base and a bonnet that is attachable or attached to the base. The base includes a base lens and the bonnet includes a bonnet lens and a bonnet ring holding the bonnet lens. The base is attached to the bonnet, the base lens and the bonnet lens are arranged so that they are movable relatively to each other and the bonnet lens is attached to the bonnet ring. The method comprises a step of compression forces being applied to the bonnet ring and the base moving the bonnet lens and the base lens towards each other.

Achievable advantages of this aspect of the invention include those discussed above with regard to the first aspect of the invention.

Finally, in a seventh aspect of the invention, a method of assembling an accommodative intraocular lens system suitable for implantation in capsular bags of multiple sizes according to claim 26 is disclosed. The method comprises a step of providing a bonnet which has an actuator for contacting a portion of the capsular bag on one of the anterior and the posterior sides of the capsular bag. The method moreover comprises a step of providing multiple bases, wherein each base has an actuator for contacting a portion of the capsular bag on the other of the anterior and the posterior sides of the capsular bag, each base is attachable to the bonnet, and each base is shaped differently such that for each base when the base is attached to the bonnet, the length of the resulting accommodative intraocular lens as measured from the actuator of the base to the actuator of the bonnet is different to that of any accommodative intraocular lens formed with any of the other bases.

Achievable advantages of this aspect of the invention include those discussed above with regard to the fifth aspect of the invention.

To implant the accommodative intraocular lens system into the human eye, a capsulorhexis, ie, a circular hole may be cut into the anterior side of the capsular bag. After removal of the natural lens, the base and bonnet can be inserted into the emptied capsular bag, preferably by means of an injector. Preferably, the base and the bonnet are injected separately. Once injected, the surgeon may assemble the base and the bonnet to form the accommodative intraocular lens system. Preferably, the bonnet is placed on the anterior side of the capsular bag and the base is placed on the posterior side. By inserting, preferably injecting by means of an injector, the base and the bonnet separately and assembling them in the eye the advantage can be achieved that a small corneal incision, preferably an incision of less than 3 mm is sufficient for insertion. Once inserted and assembled, the system can mimic the accommodation of the natural lens for example as follows: When the ciliary muscle is contracted, the system is in its near vision or intermediate vision mode, in which the base lens and the bonnet lens are in their separating position. As the ciliary muscle relaxes for focussing on objects further apart, the capsular bag is stretched via the zonules. This causes the anterior capsule (ie, the remainder of the anterior part of the capsule after capsulorhexis) to exert an inward force on actuators of the base lens and the bonnet lens such that the base lens is pressed towards the bonnet lens. The resulting deformation of at least one of the lens faces reduces the refractive power of the system, hereby enabling focusing on objects further apart.

### PREFERRED EMBODIMENTS OF THE INVENTION

Preferred features of the invention which may be applied alone or in combination are discussed in the following and in the dependent claims.

In a preferred embodiment of the invention, when the base is attached to the bonnet, the base lens and the bonnet lens are arranged so that they are movable relatively to each other. Preferably, this motion has a component in axial direction of at least one of the base lens and the bonnet lens. Even more preferably, the base lens and the bonnet lens are arranged in succession on the same optical axis and the motion is along this optical axis.

The preferred bonnet comprises a bonnet ring to which the lens is attached. Preferably, compression forces applied to the bonnet ring and to the base can move the bonnet lens and the base lens towards each other. Thereby, when the accommodative intraocular lens system is implanted into the patient's eye, compression forces originating from the capsular bag being stretched can move the bonnet lens relatively to the base lens. As a result, advantageously, the lens can respond to the forces applied by the ciliary muscles on the bag through the zonules.

The bonnet lens of the preferred accommodative intraocular lens system is provided with a bonnet lens actuator which in the intended use of the accommodative intraocular lens, when the accommodative intraocular lens is implanted in a patient's eye, contacts the capsular bag. It is achievable that via the bonnet lens actuator the capsular bag applies a force on the bonnet lens in the posterior direction. The bonnet lens actuator preferably contacts the capsular bag on the area between the rim of the capsulorhexis and the capsule equator, more preferably between the rim of the capsulorhexis and a point near where anterior zonules attach to the capsular bag. The intended contact region of the bonnet lens actuator preferably is annular or comprises multiple contact spots arranged in an annular shape.

Advantageously, the annular contact region can improve the efficiency in the transfer of the force applied by the ciliary muscles to the lens via the capsular bag. The preferred outer diameter of the annular contact region or the annular shape comprising contact the spots is more than 4.5 mm, more preferably more than 5 mm, even more preferably more than 6 mm, even more preferably more than 7 mm.

Both the annular contact region and the diameter of the annular contact region or the annular shape comprising contact the spots, which diameter preferably is considerably greater than that of a typical capsulorhexis, contributes to increasing the tolerance of the lens mechanical performance to variations on the size and shape of the capsulorhexis. It also renders the accommodative intraocular lens system less prone to shifting in the capsular bag or to escape through the capsulorhexis into the eye anterior chamber. The diameter of the annular contact region or the annular shape comprising the contact spots preferably is less than 10 mm, more preferably less than 8 mm. Preferably the bonnet comprises a bonnet ring that surrounds the bonnet lens, an anterior portion of the bonnet ring serving as the bonnet lens actuator.

In embodiments of the invention where the bonnet comprises a bonnet ring surrounding the bonnet lens, an anterior portion of the bonnet ring preferably serves as the bonnet lens actuator. Advantageously, the bonnet ring can provide for an actuator with a large area of contact with the capsular bag. A large area of contact can significantly increase the efficiency with which the forces applied by the ciliary muscles produce the compression between the lens and base. A large area of contact can also reduce the risk of the lens escaping for the capsular bag through the capsulorhexis into the eye anterior chamber is reduced.

A preferred bonnet ring comprises at least one through hole extending from the anterior side of the bonnet to the posterior side of the bonnet. Advantageously, the through hole(s) can facilitate fluid flow in the vicinity of the bonnet. In some embodiments, the through hole(s) is/are oblong holes or slots. Preferably, there are at least two, more preferably 3, 4, 5, 6, 7, 8, 9 or 10 through holes. The through hole(s) preferably is/are provided in the bonnet ring, ie, it/they extend through the bonnet ring.

It is preferred that at least some of the through hole(s) extend to the posterior side of the bonnet from a location of the bonnet's, preferably the bonnet ring's, anterior side. Particular preferably at least some of the through hole(s) extend from a side of the bonnet ring that is directed towards the bonnet lens. It is achievable that this arrangement of the hole(s) can provide for fluid flow between outside the capsular bag through the capsulorhexis and the hole(s) into the capsular bag and/or into the accommodative intraocular lens system.

Preferably, at least some of the through hole(s) extend from the anterior side of the bonnet to the posterior side of the bonnet to a location on the posterior side of the bonnet, preferably the bonnet ring, that is adjacent to a space between the bonnet lens and the base lens when the bonnet is attached to the base to form the accommodative intraocular lens system. Advantageously, this arrangement of the hole(s) can provide for fluid flow to and from the space between the bonnet lens and the base lens.

The base lens of a preferred accommodative intraocular lens system is provided with a base lens actuator which in the intended use of the accommodative intraocular lens, when the accommodative intraocular lens is implanted in a patient's eye, contacts the capsular bag. Advantageously, via the base lens actuator the capsular bag can apply a force on the base lens in the anterior direction.

In a preferred embodiment, the base lens is provided with a foot on its posterior side. The preferred foot is attached to the base lens. Advantageously, the foot can contribute to the lens system lying more stably in the capsular bag. It is preferred that the foot extends in posterior direction from the base, preferably from the base lens. It is an achievable advantage of this embodiment that the foot, preferably the length of the foot in the anterior direction, can be chosen so that the accommodative intraocular lens system properly fits inside the capsular bag. This will be discussed in more detail further below. Moreover, advantageously, in some embodiments the foot can help to prevent the base of the lens from becoming overgrown with tissue. Overgrowth is a known problem with intraocular lenses.

It is moreover preferred that a posterior part of the foot serves as the base actuator. As a result, preferably, when the base is attached to the bonnet, compression forces applied to the bonnet lens, preferably via the bonnet ring, and to the foot can move the base lens and the bonnet lens towards each other. This can further contribute to the lens system lying more stably in the capsular bag.

The length of the foot preferably is more than 0.4 mm, more preferably more than 0.7 mm, more preferably more than 1 mm. The length is measured from the location where the foot is attached to the base. The length of the foot preferably is less than 5 mm, more preferably less than 4 mm, more preferably more than 3 mm. A preferred diameter of the annular end of the foot that serves as the base actuator is more than 4.5 mm, more preferably more than 5.0 mm, even more preferably more than 6 mm, even more preferably more than 7 mm. The diameter of the annular end of the foot that serves as the base actuator preferably is less than 10 mm, more preferably less than 8 mm.

The preferred foot is a tubular ring that, more preferably, is tapered such that the tubular diameter increases in the posterior direction. The increase in diameter can provide for a larger diameter of an annular contact region between the posterior end of the foot acting as an actuator, and the capsular bag.

It is preferred that foot comprises at least one through hole extending from an outside of the foot to an inside of the foot. Such through hole can facilitate a flow of fluid in the vicinity of the base. In particular, it can provide for an exchange of fluid to and from the space inside the foot between the base lens and a face of the posterior side of the capsular bag that is directed towards the posterior side of the base lens. Also advantageously, the through holes can aid the removal of an ophthalmic viscosurgical device (OVD) from behind the accommodative intraocular lens system during cataract surgery.

In a preferred intraocular lens system, at least one of the base and the bonnet is provided with elastic webs that render the bonnet lens movable relatively to the base lens, wherein the preferred motion has a component in axial direction of at least one of the base lens and the bonnet lens. The webs preferably extend between the base collar and the base lens. Preferably, there are more than two webs, for example 3, 4, 5, 6, 7, 8, 9, 10, 11, 12 webs. A greater number of webs can render the base lens more stable in the base collar. The preferred webs are narrow and thin enough to bend elastically when the bonnet and the base are pressed against each other. In the case of 4 to 12 webs, for example, a preferred web is between 0.15 mm and 0.5 mm thick, about 0.2 mm to 0.5 mm wide and between 0.3 and 1.5 mm long.

Preferably, the webs are separated by through holes that extend from the anterior side of the base to the posterior side of the base. The preferred through holes are oblong holes or slots that have the general form of an angular section of a ring. Thereby, advantageously, in the case of a circular outer rim of the base lens, the through holes follow the shape of the outer rim. In the case of 4 to 12 webs, for example, a preferred slot is between 0.6 and 5 mm long and between 0.3 and 1.5 mm long wide. The through holes allow fluid to flow between the inner of the capsular bag and the space between the bonnet lens and the base lens.

It is preferred that at the interface of at least one of the webs with the base or the bonnet a notch is provided. Preferably, such notch is provided at the interface of each of the webs with the base or the bonnet. The preferred notch is an angular segment of a ring. Particularly preferably, the notch is a ring that passes through the interface of each of the webs with the base or the bonnet. This circular notch preferably surrounds the base lens or the bonnet lens. It is an achievable advantage of a notch that it can contribute to setting the restoring force of the web during compression to an appropriate value. Also advantageously, the notch can contribute to concentrating strain in order to protect the base lens from unwanted deformations during compression. Such protection from deformation can increase the optical of the base lens, in particular in a compressed state of the lens system. A preferred notch is less than 0.8 mm deep, preferably less than 0.4 mm, more preferably less than 0.2 mm, for example 0.17 mm deep. A preferred notch is more than 0.05 mm, preferably more than 0.10 mm, more preferably more than 0.15 mm deep.

Preferably, the bonnet ring and the base collar can be mated with each other. Thereby, advantageously, the bonnet ring can be attached to the base collar. For such mating, the base collar and the bonnet ring preferably have complementary shapes. Preferably, one of the bonnet ring and the base collar has at least one projection that extends outwards in the direction of the other one of the two components. Particularly preferably, the projection(s) is/are configured to latch into the other component. For this purpose, the other component preferably is provided with a groove, more preferably an annular groove with which the projection(s) can engage. Preferably, one projection is annular. Alternatively or in addition, there may be multiple discrete projections, which, preferably, are evenly spaced.

Apart from the bonnet ring's potential role in moving the bonnet lens and the base lens relatively to each other, and attaching the bonnet to the base, it is achievable that the bonnet ring contributes to protecting the bonnet against being overgrown with tissue. Overgrowth is a known problem with intraocular lenses.

A preferred base collar has an outer diameter greater than 5 mm, preferably greater than 7 mm, more preferably greater than 8 mm, for example 8.5 mm. A preferred base collar has a diameter of less than 12 mm, preferably less than 10 mm, more preferably less than 9 mm. The annular groove preferably is directed inwardly, ie, towards the optical axis, and towards the bonnet, ie, in the anterior direction. A preferred groove is v-shaped. The preferred opening angle of the v is less than 90°, preferably less than 80°, for example 70°. The preferred opening angle of the v is greater than 50°, preferably more than 60°, more preferably 65° or greater. A first of the two walls of the v-shaped groove preferably extends in a plane that is perpendicular to the optical axis. A second of the two walls preferably is a tubular wall extending towards the bonnet, ie, in the anterior direction. The tubular wall preferably is tapered as it extends in the anterior direction. The width of the groove, ie, how far the groove extends horizontally from the tip of the groove wall closest to the base centre up to the bottom of the groove, preferably is greater than 0.100 mm, preferably greater than 0.150 mm, for example 0.185 mm. The width of the groove preferably is less than 0.300 mm, preferably less than 0.250 mm. In the context of the present invention, the bottom of the groove, when viewed in a cross-section, is its inner point furthest to the base centre. The inner diameter of the grove as measured at the bottom of the groove preferably is 7 mm or greater, preferably 7.5 mm or greater, for example 7.7 mm. It preferably is 8.5 mm or smaller, more preferably 8 mm or smaller. The preferred projection on the bonne ring is directed outwardly, ie, away from the optical axis. It preferably is ring shaped and complementary to the groove in size and shape so that it fits snuggly into the groove.

It is an achievable advantage of the mating of the base collar and the bonnet ring that the bonnet can be attached to the base. Preferably, when the bonnet is attached to the base, an anterior face of the base lens is located opposite a posterior face of the bonnet lens. More preferably the base lens and the bonnet lens are arranged in parallel planes, particularly preferably they are arranged on a common central axis. In the context of the present invention, the central axis of an optical component such as a lens refers to an axis that extends through the centre perpendicularly to the plane of the optical component; it typically coincides with the optical axis.

The base lens and the bonnet lens preferably are arranged such that they can assume a contact position in which the contact area has a diameter of at least 1 mm, more preferably at least 1.5 mm, more preferably at least 1.75 mm, for example 2 mm. A sufficiently large contact area is important for achieving a sufficiently clear image when the base lens and the bonnet lens are pressed against each other. The contact area preferably has a circular circumference, the circle most preferably coinciding with the centres of the base lens and/or the bonnet lens. Preferably, contact area has a diameter of less than 5 mm, more preferably less than 3.5 mm, even more preferably less than 2.5 mm.

In a preferred accommodative intraocular lens system, at least one of the bonnet and the base, preferably at least one of the bonnet lens and the base lens, comprises a stop that protrudes from the one towards the other of the bonnet and the base. The stop can abut against the other of the bonnet (lens) and the base (lens) in order to limit the extent by which the anterior face of the base lens and the posterior face of the bonnet lens can be moved towards each other. With the stop attached to one of the bonnet and the base, it is achievable that when the stop is touches of the other of the bonnet and the base, it can limit the force with which the bonnet lens and the base lens are pressed against each other. In other words, the stop can resist to further unwanted compression. This way, it can be achieved that in the compressed state, the bonnet and the base lens are pressed against each other with a well-defined force that does not exceed a certain limit. The well-defined force can entail a well-defined deformation of the bonnet lens and the base lens. In other words, the stop can prevent an over-compression of the lenses.

In a preferred embodiment of the invention, in the contact position the compression force with which the posterior face of the bonnet lens presses against the anterior face of the base lens does not exceed 10 gF (gram-force), more preferably 7 gF, even more preferably 5 gF. It is an achievable advantage of this embodiment of the invention that the compression force is small enough to be produced reliably by the capsular sac in the relaxed state of the ciliary muscle. Preferably, the force required to press the posterior face of the bonnet lens against the anterior face of the base lens such that the stop attached to one of the bonnet and the base is more than 0.5 gF, more preferably 1 gF, even more preferably 2 gF. The minimum force ensures that the accommodative intraocular lens system does not inadvertently changes form the near view to the far view mode.

The preferred stop is an elevated ring or comprises multiple elevated spots arranged in an annular shape. The stop preferably extends along the outer edge of the base lens or the bonnet lens. The stop preferably is elevated by more than 5 μm, more preferably by more than 10 μm. It is elevated by preferably less than 40 μm, more preferably by less than 20 μm. A preferred stop is more than 20 μm wide, more preferably more than 40 μm. If preferably is less than 0.5 mm wide, preferably less than 150 μm.

Preferably, when the base is attached to the bonnet, the base lens and the bonnet lens are movable relatively to each other between a separating position in which the anterior face of the base lens and the posterior face of the bonnet lens are spaced apart from each other, and a contact position in which the anterior face of the base lens and the posterior face of the bonnet lens press against each other forming a contact area.

It is achievable that in the separating position, the accommodative intraocular lens system has a different refractive power than in the contact position. Preferably, the change of optical power is a result of a deformation at least one of the anterior face of the base lens and the posterior face of the bonnet lens as they press against each other forming a contact area.

Preferably, in the separating position, in which the anterior face of the base lens and the posterior face of the bonnet lens are spaced apart from each other, the refractive power of the accommodative intraocular lens system is higher than in the contact position, in which the anterior face of the base lens and the posterior face of the bonnet lens press against each other. This embodiment of the invention exploits the effect that in the contact area, the refractive powers of the anterior face of the base lens and the posterior face of the bonnet lens vanish. While, typically, this effect is partly reversed due to a deformation of the posterior face of the base lens and the anterior face of the bonnet lens as a result of the lenses pressing against each other, it can be achieved that there is a net reduction of refractive power in accommodative intraocular lens system. Accordingly, in this preferred embodiment of accommodative intraocular lens system, the separating position corresponds to the accommodated state of the natural lens, ie, the state in which the eye is focused on a point close to the eye. The contact position, in contrast, corresponds to an unaccommodated state of the natural lens, ie, the state in which the eye is focused on a point at a greater distance.

It is preferred that in the separating position, the gap between the posterior surface of the optics of the lens and the anterior surface of the optics of the base component is greater than 0.1 mm, preferably greater than 0.2 mm, more preferably greater than or equal to 0.3 mm. Advantageously, the gap can contribute to preventing a stiffening or a reduction in size of the capsular bag from interfering with the functionality of the accommodative intraocular lens system. This embodiment of the invention exploits that the gap between the base lens and the bonnet lens can serve as a buffer to compensate to some extent for the capsule bag becoming stiffer and smaller. In other words, while the stiffening or reduction in size of the capsular bag may reduce the gap between the base lens and the bonnet lens, the lenses can—as long as the stiffening or reduction in size does not exceed a certain degree—still assume the defined state of separation. Preferably, in the separating position the gap between the posterior surface of the optics of the lens and the anterior surface of the optics of the base component is smaller than 1 mm, preferably smaller than 0.5 mm, more preferably smaller than 0.4 mm. Too great a gap can compromise the ability of the lenses to reach the contact position or press against each enough to achieve a sufficiently large contact area and/or a sufficiently large change in refractive power.

Preferably, one of the base lens and the bonnet lens, more preferably the base lens, has zero refractive power. Note that, as defined above, the refractive power is measured in the relaxed state of the lens. Preferably, the other of the base lens and the bonnet lens has a positive refractive power, preferably greater than 0 dioptre, more preferably greater than 5 dioptre. Preferably, the refractive power of the other of the base lens and the bonnet lens is smaller than 35 dioptre, more preferably smaller than 30 dioptre. It is an achievable advantage of this range of refractive power that the accommodative intraocular lens system can replace the natural lens of the eye, ie, it can serve as a pseudophakic intraocular lens. The preferred lenses are bodies of revolution or rotationally symmetrical.

In a preferred embodiment of the invention, at least one face of at least one of the base lens and the bonnet lens of the accommodative intraocular lens system is flat or concave, preferably flat. It is an achievable advantage of the flat face of the base lens or the bonnet lens that it has zero refractive power when the lens is relaxed, and therefore its effect on the accommodative intraocular lens system is easier to predict, even in a situation in which the lens bearing the flat face is deformed due to compression—for example when the anterior face of the base lens and the posterior face of the bonnet lens press against each other—and therefore is no longer flat. It is preferred that at least one of the anterior face of the base lens and the posterior face of the bonnet lens is flat or concave. More preferably, at least one face of at least one of the base lens and the bonnet lens, even more preferably at least one of the anterior face of the base lens and the posterior face of the bonnet lens, is convex. In a particularly preferred embodiment, one of the anterior face of the base lens and the posterior face of the bonnet lens is flat or concave while the other is convex. In one embodiment of the invention, the anterior face of the base lens is flat or concave while the posterior face of the bonnet lens is convex.

Preferably, the thickness of the base lens and the thickness of the bonnet lens is greater than 0.8 mm, more preferably greater than 1 mm, more preferably greater than 1.2 mm. Preferably, the thickness of the base lens and the thickness of the bonnet lens is less than 1.7 mm, more preferably less than 1.5 mm, more preferably less than 1.3 mm.

To properly fit the capsular bag, the accommodative intraocular lens system preferably has a thickness—as measured from the actuator the base to the actuator of the bonnet—of more than 2.6 mm, preferably more than 2.8 mm, more preferably more than 3.0 mm. The thickness of the preferred accommodative intraocular lens system is less than 6 mm, preferably less than 5 mm, more preferably less than 4.5 mm.

A preferred set has at least two bases, more preferably at least 3, 4, 5, 6, 7, 8, 9 or 10 bases. In a preferred set for assembling an accommodative intraocular lens system, the lengths of the accommodative intraocular lens system, depending on which of the bases has been chosen, range between 2.6 mm and 6 mm, preferably between 2.8 mm and 5 mm, more preferably between 3.0 mm and 4.5 mm.

It is preferred that at least two, more preferably all bases of the set comprise a foot. Preferably at least two, of the multiple bases differ in the length of their feet, more preferably the length of the foot of each base with a foot is different to the length of the foot of each other base with a foot. This has the achievable advantage that the foot, preferably the length of the foot, can be chosen so that the hybrid lens properly fits inside the capsular bag. In a preferred set for assembling an accommodative intraocular lens system, the lengths of the feet, depending on which of the bases has been chosen, range between 0.4 mm and 2.5 mm, preferably between 0.7 mm and 2.0 mm, more preferably between 1 mm and 1.8 mm.

The preferred method of assembling an accommodative intraocular lens system suitable for implantation in capsular bags of multiple sizes comprises, in addition to the steps of providing the bonnet and the bases the steps of selecting a base. The base preferably is selected based on a measurement of the size of the capsular bag of a patient's eye. Suitable method of measuring the size of the capsular bag is the UBM (ultrasound biomicroscopy) method disclosed in C. J. Pavlin, K. Harasiewicz, M. D. Sherar, F. S. Foster; Clinical use of ultrasound biomicroscopy; Ophthalmology, 98 (1991), pp. 287-295. From the capsular bag equatorial diameter (C_{D}), circumferential length of the anterior capsule surface (C_{i_ant}) and circumferential length of the posterior capsule surface (C_{i_post}) thus obtained, the ideal thickness Lₜ of the accommodative intraocular lens system can be determined by means of the formula

$${L}_{t} = \frac{1}{2} ( \sqrt{{C}_{{l}_{-} a n {t}^{2}} - {C}_{D}^{2} - 2 {L}_{{w}_{-} a n t} ( {C}_{{l}_{-} a n t} - {C}_{D} )} + \sqrt{{C}_{{l}_{-} p o s {t}^{2}} - {C}_{D}^{2} - 2 {L}_{{w}_{-} p o s t} ( {C}_{{l}_{-} p o s t} - {C}_{D} )} ) + 0.5 ,$$

where L_{w_ant} is the diameter of the circle defined by the top of the bonnet actuator and L_{w_post} is the diameter of the circle defined by the bottom of the base actuator. From the bases of the set, preferably the base is chosen that, when combined with the bonnet, achieves a thickness of the accommodative intraocular lens system that comes closest to Lₜ. The method moreover preferably includes a step of implanting the lens into the eye the size of the capsular bag of which was estimated or measured in the previous step.

As to the orientation of the accommodative intraocular lens system when the accommodative intraocular lens is implanted in a patient's eye, it is preferred that the bonnet is oriented towards the cornea and the base is oriented towards the vitreous body. Preferably, the capsulorhexis is on the side of the capsular bag that is oriented towards the cornea and the bonnet lens actuator is in contact with the interior of this side of the capsular bag, while the base lens actuator is in contact with the interior of the side of the capsular bag that is oriented towards the vitreous body.

The preferred base lens is made of a uniform material. Preferably, the base lens is formed integrally with the base collar and/or the base lens actuator and/or the webs. More preferably, the entire base is formed integrally, most preferably of a uniform material. This greatly simplifies the manufacture of the base.

Similarly, the preferred bonnet lens is made of a uniform material. Preferably, the bonnet lens is formed integrally with the bonnet ring. More preferably, the entire bonnet is formed integrally, most preferably of a uniform material. This greatly simplifies the manufacture of the bonnet.

At least one of the base lens and the bonnet lens, are made of an elastic material. Suitable materials include plastic materials like, for example, hydrogel, silicone, soft acrylic polymers, or combinations thereof. If not denoted otherwise, the term “polymer” as used herein includes the term “copolymer”, ie, a polymer that has been produced based on at least two different monomers. In a preferred embodiment the lens, the base component or both are made from an acrylic-based polymer or an acrylic-based copolymer, more preferably from a soft acrylic-based polymer or a soft acrylic-based copolymer, in particular from a copolymer of hydroxyethyl methacrylate and methyl methacrylate. It is an achievable advantage of acrylic-based polymer and copolymer materials that they are elastic and have a high biocompatibility. The preferred material is hydrophilic. Preferably, both the base lens and the bonnet lens are made of an elastic material, more preferably an acrylic-based polymer or an acrylic-based copolymer.

Preferably, the base and the bonnet are made from the same plastic material. This has the advantage that both components show the same elastic behaviour, rendering the behaviour of the accommodative intraocular lens system easier to predict. Also, advantageously, their identical optical density causes the refractive power in the contact area of the base lens and the bonnet lens to vanish. In an alternative embodiment, the base is made from a first material and the bonnet is made from a second material, the second material having a different elastic modulus than the first material. Using different materials for the base and for the bonnet provides an additional degree of freedom to the design of the accommodative intraocular lens system as intrinsic properties of the materials such as stiffness can be chosen independently for both components. Preferably, the optical density of the first material and the second material is chosen to be similar or identical.

The preferred elastic modulus of the at least one of the base lens and the bonnet lens is greater than 1.5 MPa (megapascal), preferably greater than 1.8 MPa. It is an achievable advantage of materials with an elastic modulus of more than 1.5 MPa that they aid in achieving a significant difference in power between the separating position and the contact position are easier to assemble in the human eye during surgery. The preferred elastic modulus of the at least one of the base lens and the bonnet lens is smaller than 4 MPa, preferably smaller than 3.2 MPa. It is an achievable advantage of a material with an elastic modulus of less than 4 MPa that it provides sufficient elasticity to allow for a sufficiently large contact area and a sufficient extent of deformation to aid in achieving a significant difference in power between the separating position and the contact position.

In the context of the present invention, the terms “elastic modulus”, also referred to as the “modulus of elasticity” and “Young's modulus”, denotes the ratio of a stress in a body to a corresponding strain resulting from the deformation of the body caused by the application of stress. The elastic modulus is the slope of the stress-strain curve in the range of linear proportionality of stress to strain. The greater the modulus, the stiffer the material, or the smaller the elastic strain that results from the application of a given stress. The elastic modulus is measured at a temperature of 35° C. (Celcius), and a hydration of the material of 26%, provided that the material is hydrophilic, or as appropriate for the selected material.

### BRIEF DESCRIPTION OF THE DRAWINGS

In the following, further preferred embodiments of invention are illustrated by means of examples. The invention is not limited to these examples, however.

The drawings schematically show:

FIG. 1A perspective view from the anterior side onto an assembled accommodative intraocular lens system according to the invention;

FIG. 2A cross-sectional view of the assembled accommodative intraocular lens system of FIG. 2;

FIG. 3A perspective view from the anterior side onto the bonnet of the accommodative intraocular lens system according to FIG. 1;

FIG. 4A perspective view from the anterior side onto the base of the accommodative intraocular lens system according to FIG. 1;

FIG. 5A perspective view from the posterior side onto the base of the accommodative intraocular lens system according to FIG. 1; and

FIG. 6A cross-sectional view of the posterior face of the bonnet lens, the optical axis being in the cross-sectional plane.

### DETAILED DESCRIPTION OF AN EMBODIMENT OF THE INVENTION

In the following description of preferred embodiments of the invention, identical reference numerals refer to identical or similar components. For clarity, in the case of multiple identical parts in the figure generally only one of these parts is provided with a reference numeral.

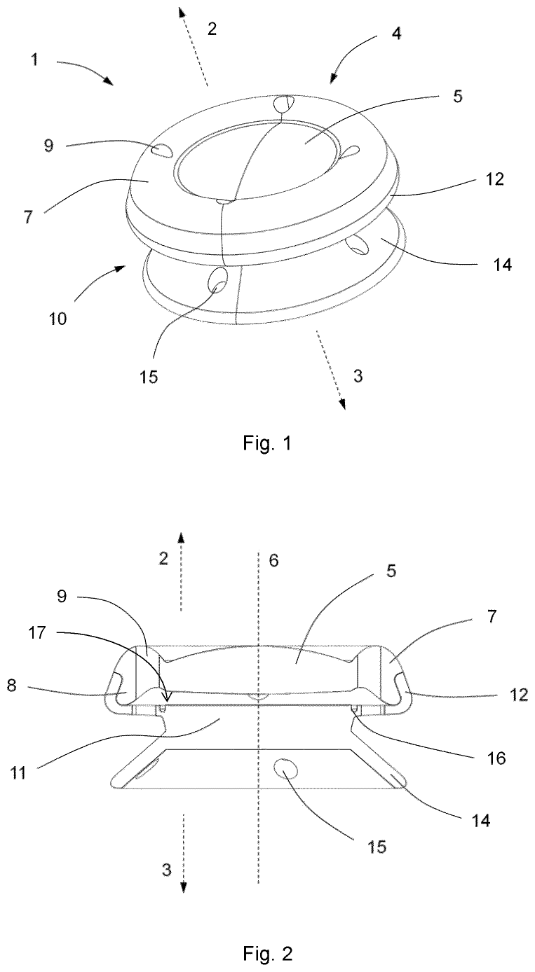

FIG. 1 shows a perspective view from the anterior side onto an accommodative intraocular lens system according to the invention. FIG. 2 shows a cross-sectional view of the system through a central plane symbolized by the solid line in FIG. 1. The accommodative intraocular lens comprises a bonnet 4 and a base 10. The bonnet 4 is oriented towards the cornea 2 of the human eye, whereas the base 10 is oriented towards the vitreous body 3. The arrows 2 and 3 denote the respective directions in the Figures.

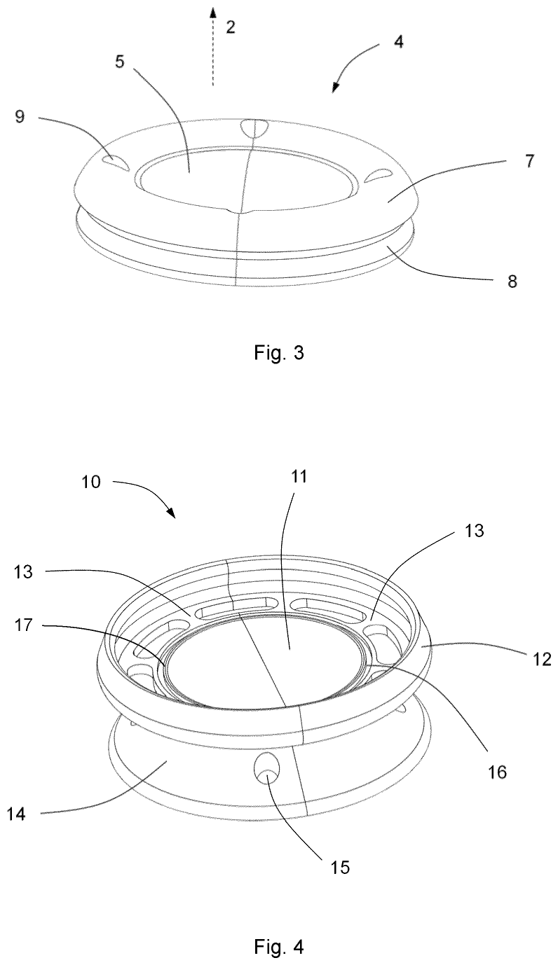

FIG. 3 shows a perspective view from the anterior side onto the bonnet 4 of the accommodative intraocular lens system. The bonnet 4 has a bonnet lens 5 with a positive refractive power optics with an optical axis 6. A bulging bonnet ring 7 annularly surrounds the central bonnet lens 5 on its circumference, the bulging bonnet ring 7 and the bonnet lens being firmly connected to each other. In the exemplary embodiment shown the bonnet is one-piece in the sense that the bonnet lens 5 and the bonnet ring 7 are integrally connected and made from the same material.

The anterior part of the bonnet ring 7 serves as a bonnet actuator, which in the intended use of the accommodative intraocular lens, when the accommodative intraocular lens is implanted in a patient's eye, contacts the capsular bag. The apex of the bonnet ring 7 in the anterior direction forms a ring, the diameter of which is between the outer diameter of the bonnet ring 7 and the inner diameter of the bonnet ring 7, which corresponds to the outer diameter of the bonnet lens 5 of the bonnet 4. The bonnet actuator contacts the capsular bag in an annular contact region around this ring-shaped apex, applying a compression force to the bonnet lens 5 via the bonnet ring 7 when the capsular bag is stretched due to the ciliary muscle of the eye being relaxed.

The posterior part of the bonnet ring 7 has a circumferential projection 8 that extends outwards in the posterior direction 3 and that extends beyond the posterior face of the lens 5 of the bonnet 4 in the posterior direction of the optical axis 8. This projection serves to attach the bonnet to the base. The bonnet ring 7 contains four circular through holes 9 that extend from the anterior face of the bulging bonnet ring 7 to its posterior face. In an alternative embodiment (not shown in the figures), the bonnet ring is identical to that shown in FIG. 3 but has eight rather than four through holes. The through holes 9 are located on the inner side of the bonnet ring, ie, the side of the bonnet ring that is directed towards the bonnet lens so that fluid can flow from the outside the capsular bag through the capsulorhexis and the hole 9 into the space between the bonnet lens 5 and the base lens 11. The holes 9 are equally spaced along a circumferential line of the bulging ring 7.

The base 10 comprises a base lens 11 and an annular base collar 12, which surrounds the base lens. The annular collar 12 extends inwards in the anterior direction 2 thereby forming, on the inner side, an annular groove that can engage with and hold the circumferential projection 8 of the bonnet ring 7.

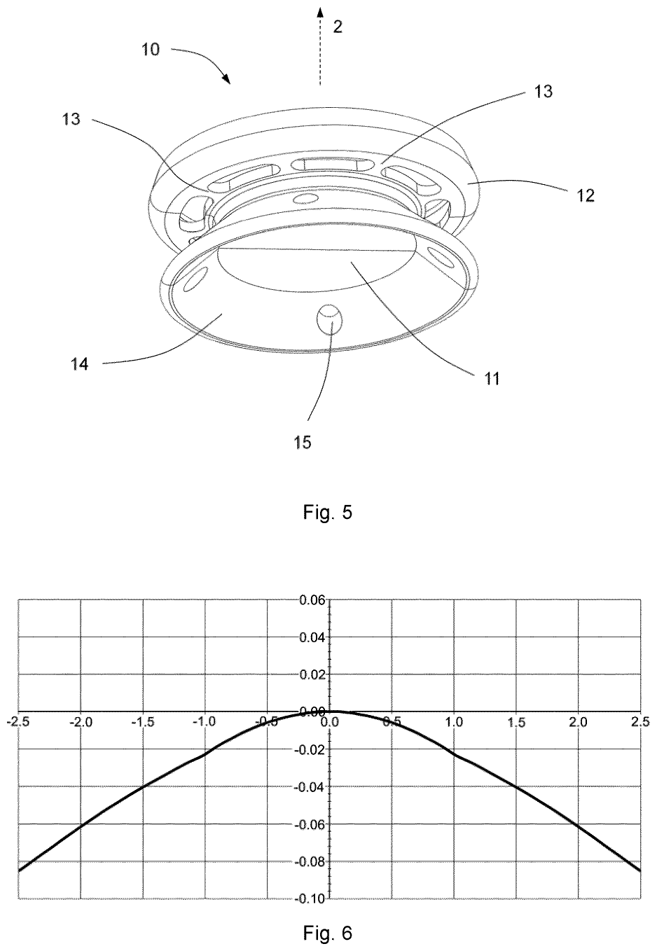

The base collar 12 is connected to the base lens 11 via eight elastic webs 13. FIGS. 4 and 5 show perspective views onto the base 10 of the accommodative intraocular lens system from the anterior side and from the posterior side, respectively. The webs 13 are separated by oblong through holes that have the general form of an angular section of a ring. An annular notch 16 is provided between the base lens 11 and the webs 13 of the base 10. Moreover, the base lens is provided with a stop 17 (best seen in FIG. 4) that protrudes in anterior direction towards the bonnet lens 5 and can contact the bonnet lens 5 to limit the force with which the base lens 11 presses against the bonnet lens 5.

The base 10 further comprises a cone-shaped tubular foot 14 extending from the base lens 11 of the base 10 outwards in the posterior direction. The posterior end of the foot 14 serves as a base actuator contacting the capsular bag in an annular contact region and applying a compression force to the base lens 11 when the capsular bag is stretched due to the ciliary muscle of the eye being relaxed. The foot contains four through holes 15 that are equally spaced along the circumference of the foot 14.

The bonnet 4 and the base 10 are attached to each other. Size and shape of the projecting portion 8 of the bonnet 4 and the annular collar 12 of the base 10 are configured such that the projecting portion 8 latches into the annular collar 12.

In the unaccommodated state for distance vision the posterior face of the bonnet lens 5 contacts the anterior face of the base lens 11, whereas in the accommodated state for near vision mode, ie, if no external compression force are applied to the exemplary accommodative intraocular lens system, the posterior face of the bonnet lens 5 and the anterior face of the base lens 11 are spaced apart from each other. FIG. 2 shows the accommodative intraocular lens system in the accommodated state.

### Example

An accommodative intraocular lens system according to FIGS. 1 to 6 has been manufactured and tested for its properties. Bonnet and base were both manufactured from a copolymer of hydroxyethyl methacrylate and methyl methacrylate. The material had the following properties: swell factor of 1.13; water content at 35° C. of 26%; refractive index at 35° C. hydrated of 1.460; tensile strength of 2.5 MPa; elastic modulus of 3.0 MPa and elongation to break of 250%. The bonnet and the base were both manufactured as one-piece each.

The total diameter of the bonnet was 8.5 mm. The thickness of bonnet lens inside the bonnet ring was 1.2 mm. The ring that is formed by the apex of the bulging bonnet ring in the anterior direction had a diameter of 6.6 mm. The diameter of each of the holes in the bulging bonnet ring was 0.6 mm.

The opening angle of the groove, ie, the angle between the central portion and the annular collar of the base component was 70°. The annular collar extended from the central portion of the base component in the anterior direction for 0.9 mm. The thickness of the annular collar in its radial direction was 0.3 mm. The webs connecting the annular collar with the central portion of the base component had a width in the circumferential direction of 0.3 mm each. The distance between two neighbouring webs and thus the lengths of the oblong through holes between these webs was 2.1 mm each. The thickness of the webs at their radially outward end was 0.252 mm and the thickness of the webs at their radially inward end was 0.285 mm; thus, ratio of the thickness of the webs at their radially outward end to the thickness of the webs at their radially inward end was 0.88. The depth of the annular notch between the base lens and the webs of the base component was 0.17 mm.

The foot was 1.6 mm long and the foot's annular actuator for contacting the posterior part of the capsular bag had a diameter of 8 mm. The four holes in the foot had a diameter of 0.6 mm each. The annular stop extends from the base lens 13 μm in the anterior direction and is 70 μm wide.

The thickness of the base lens was 1.3 mm. The base lens had zero refractive power. In the accommodated state, the base lens and the bonnet lens were separated by 0.3 mm, and the accommodative lens system is 3.85 mm thick as measured from the contact area of the base to the contact area of the bonnet. In the unaccommodated state, the posterior face of the bonnet lens and the anterior face of the base lens contacted each other. The contact area had a diameter of 2 mm. The lens had a refractive power of 24 dioptre for near vision. The difference in power between the separation position and the compressed position was determined to be approximately 40%. The compression force onto the lens system was around 4.5 gram-force.

FIG. 6 shows a cross section of the posterior face of the bonnet lens. The optical axis is in the cross-sectional plane. The curvature has a discontinuity at a radius of 1 mm. The discontinuity causes the lens surface to comprise of a circular inner zone with of a radius of 1 mm, and an annular outer zone surrounding and adjacent to the inner zone. The lens face's radius of curvature in the inner zone is 20.6 mm, and the lens face's radius of curvature in the outer zone is 34.25 mm. The smaller radius of curvature in the outer zone provides the lens face with a negative spherical aberration.

In an alternative example not shown in the figures, the outer zone differs from that described above in that is aspheric so that the lens presents a net negative spherical aberration of 0.238 μm.

An exemplary set of for assembling an accommodative intraocular lens system comprises a bonnet as described above, a first base a described above and two additional bases that differ from the first base only in that the length of their foot was 1.0 mm and 1.3 mm, respectively. This renders the assembly of the bonnet with the bases 3.2 mm, 3.5 and 3.8 mm thick. The size of a capsular bag of an eye of patient was measured with the UBM (ultrasound biomicroscopy) method disclosed in C. J. Pavlin, K. Harasiewicz, M. D. Sherar, F. S. Foster; Clinical use of ultrasound biomicroscopy; Ophthalmology, 98 (1991), pp. 287-295. With such method, the anterior capsule length (C_{i_ant}), the posterior capsule length (C_{i_post}) and the capsule equatorial diameter (C_{D}) were found to measure 10.4 mm, 12.0 mm and 9.4 mm respectively. From this, using the formula

$${L}_{t} = \frac{1}{2} ( \sqrt{{C}_{{l}_{-} a n {t}^{2}} - {C}_{D}^{2} - 2 {L}_{{w}_{-} a n t} ( {C}_{{l}_{-} a n t} - {C}_{D} )} + \sqrt{{C}_{{l}_{-} p o s {t}^{2}} - {C}_{D}^{2} - 2 {L}_{{w}_{-} p o s t} ( {C}_{{l}_{-} p o s t} - {C}_{D} )} ) + 0.5 ,$$

where L_{w_ant} is the diameter of the circle defined by the top of the bonnet actuator and L_{w_post} is the diameter of the circle defined by the bottom of the base actuator. The ideal thickness of the lens was determined to be Lₜ=3.95 mm, and accordingly, the base with a 1.6 mm long foot was chosen, because it entails a thickness of the accommodative intraocular lens system of 3.85 mm, which is closest to Lₜ.

The features as described in the above description, claims and figures can be relevant individually or in any combination to realise the various embodiments of the invention.

### REFERENCE NUMERALS

1 accommodative intraocular lens system
2 anterior side
3 posterior side
4 bonnet
5 bonnet lens
6 optical axis
7 bonnet ring
8 projection
9 hole in bonnet ring
10 base
11 base lens
12 base collar
13 web
14 foot
15 hole in foot
16 notch
17 stop

1. An accommodative intraocular lens system comprising a base and a bonnet that is attachable or attached to the base, wherein the base includes a base lens and the bonnet includes a bonnet lens and a bonnet ring holding the bonnet lens, wherein when the base is attached to the bonnet, the base lens and the bonnet lens are arranged so that they are movable relatively to each other, and wherein the bonnet lens is attached to the bonnet ring such that when the base is attached to the bonnet, a compression force applied to the bonnet ring and to the base can move the bonnet lens and the base lens towards each other.

2. The accommodative intraocular lens system of claim 1, wherein the bonnet lens is provided with a bonnet lens actuator which in the intended use of the accommodative intraocular lens, when the accommodative intraocular lens is implanted in a patient's eye, contacts the capsular bag.

3. The accommodative intraocular lens system of claim 2, wherein the bonnet comprises a bonnet ring surrounding the bonnet lens, and an anterior portion of the bonnet ring serves as the bonnet lens actuator.

4. The accommodative intraocular lens system of claim 2, wherein the portion of the bonnet lens actuator that in the intended use of the accommodative intraocular lens, when the accommodative intraocular lens is implanted in a patient's eye is in contact with the capsular bag, has a diameter of more than 4.5 mm.

5. The accommodative intraocular lens system of claim 1, wherein the bonnet ring comprises at least one through hole extending from the anterior side of the bonnet to the posterior side of the bonnet.

6. The accommodative intraocular lens system of claim 1, wherein a base lens is provided with a base lens actuator which in the intended use of the accommodative intraocular lens, when the accommodative intraocular lens is implanted in a patient's eye, contacts the capsular bag.

7. The accommodative intraocular lens system of claim 6, wherein the base lens is provided with a foot on its posterior side, the foot being attached to the base lens, the foot or a part of the foot serving as the base actuator.

8. An accommodative intraocular lens system comprising a base and a bonnet that is attachable or attached to the base, wherein the base includes a base lens and the bonnet includes a bonnet lens, wherein when the base is attached to the bonnet, the base lens and the bonnet lens are arranged so that they are movable relatively to each other, and wherein the base lens is provided with a foot.

9. The accommodative intraocular lens system of claim 7, wherein the foot is attached to the base lens such that when the base is attached to the bonnet, compression forces applied to the foot and the bonnet lens can move the bonnet lens and the base lens towards each other.

10. The accommodative intraocular lens system of claim 7, wherein the foot is a tubular ring that is tapered such that the tubular diameter increases in the posterior direction.

11. The accommodative intraocular lens system of claim 7, wherein the length of the foot is greater than 0.4 mm.

12. The accommodative intraocular lens system of claim 7, wherein the foot comprises at least one through hole extending from an outside of the foot to an inside of the foot.

13. The accommodative intraocular lens system of claim 7, wherein at least one of the base and the bonnet is provided with elastic webs that render the bonnet lens movable relatively to the base lens.

14. The accommodative intraocular lens system of claim 13, wherein the webs are separated by through holes that extend from the anterior side of the base to the posterior side of the base.

15. An accommodative intraocular lens system comprising a base and a bonnet that is attachable or attached to the base, wherein the base includes a base lens and the bonnet includes a bonnet lens, wherein at least one of the base and the bonnet is provided with elastic webs that render the bonnet lens movable relatively to the base lens, and wherein
at the interface of at least one of the webs with the base or the bonnet a notch is provided.

16. The accommodative intraocular lens system of claim 15, wherein the base comprises a base collar surrounding and holding the base lens.

17. The accommodative intraocular lens system of claim 15, wherein the base collar and the bonnet ring can be mated with each other in order to attach the bonnet to the base so that an anterior face of the base lens is located opposite a posterior face of the bonnet lens.

18. An accommodative intraocular lens system comprising a base and a bonnet that is attachable or attached to the base, wherein the base includes a base lens and the bonnet includes a bonnet lens, and wherein when the base is attached to the bonnet, the base lens and the bonnet lens are arranged so that they are movable relatively to each other and the base lens and the bonnet lens can assume at least one relative position in which the anterior face of the base lens and the posterior face of the bonnet lens press against each other forming a contact area, and wherein
at least one of the bonnet and the base comprises a stop that protrudes from the one towards the other of the bonnet and the base and that can abut against the other of the bonnet and the base in order to limit the extent by which the anterior face of the base lens and the posterior face of the bonnet lens can be moved towards each other to press against each other.

19. The accommodative intraocular lens system of claim 18, wherein the stop is an elevated ring.

20. The accommodative intraocular lens system of claim 18, wherein the stop is higher than 0.05 mm.

21. The accommodative intraocular lens system of claim 18, wherein when the base is attached to the bonnet, the base lens and the bonnet lens are movable relatively to each other between a separating position in which the anterior face of the base lens and the posterior face of the bonnet lens are spaced apart from each other, and a contact position in which the anterior face of the base lens and the posterior face of the bonnet lens press against each other forming a contact area.

22. A set for assembling an accommodative intraocular lens system suitable for implantation in capsular bags of multiple sizes, the set comprising multiple bases and a bonnet that is attachable to each of the bases, wherein the bonnet has an actuator for contacting a portion of the capsular bag on one of the anterior and the posterior sides of the capsular bag, and each base has an actuator for contacting a portion of the capsular bag on the other of the anterior and the posterior sides of the capsular bag, and wherein each base is shaped differently such that for each base when the base is attached to the bonnet, the length of the resulting accommodative intraocular lens as measured from the actuator of the base to the actuator of the bonnet is different to that of any accommodative intraocular lens formed with any of the other bases.

23. The set of claim 22, wherein at least two of the multiple bases comprise a foot.

24. The set of claim 23, wherein at least two of the multiple bases differ in the length of their feet.

25. A method of operation of an accommodative intraocular lens system comprising a base and a bonnet that is attachable or attached to the base, wherein the base includes a base lens and the bonnet includes a bonnet lens and a bonnet ring holding the bonnet lens, the base is attached to the bonnet, the base lens and the bonnet lens are arranged so that they are movable relatively to each other and the bonnet lens is attached to the bonnet ring, wherein the method comprises a step of compression forces being applied to the bonnet ring and the base moving the bonnet lens and the base lens towards each other.

26. A method of assembling an accommodative intraocular lens system suitable for implantation in capsular bags of multiple sizes, the method comprising the steps of:
Providing a bonnet which has an actuator for contacting a portion of the capsular bag on one of the anterior and the posterior sides of the capsular bag;
providing multiple bases, wherein each base has an actuator for contacting a portion of the capsular bag on the other of the anterior and the posterior sides of the capsular bag, each base is attachable to the bonnet, and each base is shaped differently such that for each base when the base is attached to the bonnet, the length of the resulting accommodative intraocular lens as measured from the actuator of the base to the actuator of the bonnet is different to that of any accommodative intraocular lens formed with any of the other bases.
